(19) United States
(12) Patent Application Publication
(10) Pub. No.: US 2021/0353685 A1
(43) Pub. Date: Nov. 18, 2021

(54) Augmentation of Cell Therapy Efficacy by Inhibition of Complement Activation Pathways

(71) Applicant: Brain Cancer Research Institute, San Diego, CA (US)

(72) Inventors: Thomas Ichim, San Diego, CA (US); Feng Lin, San Diego, CA (US); Sandeep Pingle, San Diego, CA (US); Shashanka Ashili, Rosemount, MN (US)

(21) Appl. No.: 17/320,890

(22) Filed: May 14, 2021

Related U.S. Application Data
(60) Provisional application No. 63/024,823, filed on May 14, 2020.

(51) Int. Cl. A61K 35/28; C07K 16/18; A61K 39/395; A61K 45/06

(52) U.S. Cl. CPC A61K 35/28; C07K 16/18; C07K 2317/622; A61K 45/06; C07K 2317/24; A61K 39/3955

(57) ABSTRACT

Disclosed are means, methods and compositions of matter useful for treatment of inflammatory and/or viral mediated disease through administration of cellular populations subsequent to modulation of complement pathway. In one embodiment, patients with COVID-19 who are eligible for stem cell therapy are pretreated with modulators of complement activity in order to reduce inflammation and to augment activity of said stem cell therapy. Activity of said stem cell therapy includes protection of pulmonary cells from dysfunction/death, stimulation of regenerative/trophic activities, reduction of inflammation, and induction of immune modulation.

### CROSS-REFERENCE TO RELATED APPLICATIONS

This application claims priority to U.S. Provisional Application No. 63/024,823, titled “AUGMENTATION OF CELL THERAPY EFFICACY BY INHIBITION OF COMPLEMENT ACTIVATION PATHWAYS”, and filed May 14^{th}, 2020, which is hereby incorporated by reference in its entirety.

### FIELD OF THE INVENTION

The invention pertains to the field of treating inflammation and/or viral mediated disease through administration of cellular populations subsequent to modulation of complement pathway.

### BACKGROUND

The blood borne protein family of “Complement” was first discovered in the 1890s when it was found to aid or “complement” the killing of bacteria by heat-stable antibodies present in normal serum [1, 2]. The complement system consists of more than 30 proteins that are either present as soluble proteins in the blood or are present as membrane-associated proteins [3]. Activation of complement leads to a sequential cascade of enzymatic reactions, known as complement activation pathways, resulting in the formation of the potent anaphylatoxins C3a and C5a that elicit a plethora of physiological responses that range from chemoattraction to apoptosis. Initially, complement was thought to play a major role in innate immunity where a robust and rapid response is mounted against invading pathogens [4].

Recently it is becoming increasingly evident that complement also plays an important role in adaptive immunity involving T and B cells that help in elimination of pathogens [5]. One of the early studies demonstrating involvement of complement in adaptive immunity showed that the fifth component of the complement cascade, C5a, is capable of potentiating antigen- and alloantigen-induced T cell proliferative responses. It was found that the carboxyterminal arginine of C5a is not essential in order for C5a to enhance immune responses. C5ades Arg was found to augment the immune response to the level of C5a-mediated enhancement. The serum carboxypeptidase inhibitor, 2-mercaptomethyl-5-quanodinopentanoic acid, which prevents cleavage of the terminal arginine, allowed for assessment of the effects of C5a on in vitro immune responses in the presence of serum. It was shown that helper T cells are involved in C5a-mediated immuno-potentiation. Substitution of T cells by soluble T cell-replacing factors, (Fc)TRF, rendered lymphocyte cultures refractory to the enhancing properties of C5a [6].

In another study, flow cytometry analysis was used identify the complement 5a receptor (C5aR) on T cells. It was found that this is expressed at a low basal level on unstimulated T cells and was strikingly up-regulated upon PHA stimulation in a time- and dose-dependent manner. CD3+ sorted T cells as well as Jurkat T cells were shown to express C5aR mRNA as assessed by RT-PCR. In order for the scientists to demonstrate that C5a was biologically active on T cells, we investigated the chemotactic activity of C5a and observed that purified CD3+ T cells are chemotactic to C5a at nanomolar concentrations. Finally, using a combination of in situ hybridization and immunohistochemistry, the investigators showed that the T cells infiltrating the central nervous system during experimental allergic encephalomyelitis express the C5aR mRNA. These data suggest that innate inflammation may trigger T cell chemotaxis to areas of immunological need [7]. Complement components other than C5 are also involved in T cell activation. For example, in one study, allospecific immunoglobulin (Ig)G response was markedly impaired in C3- and C4-, but not in C5-deficient mice. This defect was most pronounced for second set responses. C3-deficient mice also demonstrated a decreased range of IgG isotypes. In contrast, there was no impairment of the allospecific IgM response. In functional T cell assays, the proliferative response and interferon-gamma secretion of recipient lymphocytes restimulated in vitro with donor antigen was decreased two- to threefold in C3-deficient mice [8].

The role of complement in host T cell mediated defenses also appears relevant. Indeed patients with complement genetic deficiencies are known to possess weaker T cell responses. In animals, a strong basic research study examined the CD8(+) T cell response in influenza type A virus-infected mice treated with a peptide antagonist to C5aR to test the potential role of complement components in CD8(+) T cell responses. It was demonstrated both the frequency and absolute numbers of flu-specific CD8(+) T cells are greatly reduced in C5aR antagonist-treated mice compared with untreated mice. This reduction in flu-specific CD8(+) T cells is accompanied by attenuated antiviral cytolytic activity in the lungs. These results demonstrate that the binding of the C5a component of complement to the C5a receptor plays an important role in CD8(+) T cell responses [9]. While the previous study demonstrated reduction in complement can compromise T cell immunity, another study demonstrated enhancement of complement augmented T cell responses. The investigators used mice deficient for decay accelerating factor (DAF), which breaks down complement. Compared with wild-type mice, DAF knockout (Daf-1(−/−)) mice had markedly increased expansion in the spleen of total and viral Ag-specific CD8+ T cells after acute or chronic LCMV infection. Splenocytes from LCMV-infected Daf-1(−/−) mice also displayed significantly higher killing activity than cells from wild-type mice toward viral Ag-loaded target cells, and Daf-1(−/−) mice cleared LCMV more efficiently. Importantly, deletion of the complement protein C3 or the receptor for the anaphylatoxin C5a (C5aR) from Daf-1(−/−) mice reversed the enhanced CD8+ T cell immunity phenotype. These results demonstrate that DAF is an important regulator of CD8+ T cell immunity in viral infection and that it fulfills this role by acting as a complement inhibitor to prevent virus-triggered complement activation and C5aR signaling [10]. Others studies have confirmed a role for various complement components in manipulation of T cell immunity [11-34].

The interaction between the innate and adaptive branches of the immune system have been previously described at several levels. For example, T cell activation of dendritic cells usually requires dendritic cells to mature in order to allow for proper antigen presentation and formation of the immunological synapse [35]. It is established that immature dendritic cells are generally tolerogenic, and induce T regulatory cells as opposed to proper T cell activation [36-82]. The process of immature dendritic cells stimulating suppressor T cells is well known in cancer, in which tumors inhibit dendritic cell maturation through production of factors such as VEGF, PGE-2, IL-10 and TGF-beta [83-86]. In the natural context, apoptotic cells possess phosphotidylserine on their surface, which maintains dendritic cells in immature states [87-98]. In contrast, during tissue damage, or infection, dendritic cells mature due to activation of receptors such as toll like receptors. Mature dendritic cells subsequent activate T cell immunity due to expression of both Signal 1 (MHC/antigen) and Signal 2 (costimulatory signals) [99]. Interestingly, some studies have shown that apoptotic bodies actually inhibit expression and/or signaling of toll like receptors [100-103].

At a basic level, complement activation is known to occur through three different pathways: alternate, classical, and lectin, involving proteins that mostly exist as inactive zymogens that are then sequentially cleaved and activated. All pathways of complement activation lead to cleavage of the C5 molecule generating the anaphylatoxin C5a and, C5b that subsequently forms the terminal complement complex (C5b-9). C5a exerts a predominant pro-inflammatory activity through interactions with the classical G-protein coupled receptor C5aR (CD88) as well as with the non-G protein coupled receptor C5L2 (GPR77), expressed on various immune and non-immune cells. C5b-9 causes cytolysis through the formation of the membrane attack complex (MAC), and sub-lytic MAC and soluble C5b-9 also possess a multitude of non-cytolytic immune functions. These two complement effectors, C5a and C5b-9, generated from C5 cleavage, are key components of the complement system responsible for propagating and/or initiating pathology in different diseases, including paroxysmal nocturnal hemoglobinuria, rheumatoid arthritis, ischemia-reperfusion injuries and neurodegenerative diseases.

Although complement has been implicated in numerous states of immunity, to one has examined the effects of complement manipulation as a means of altering efficacy of cell therapy.

### SUMMARY

Various aspects of the invention are directed to methods of enhancing efficacy of cellular therapy comprising the steps of: a) obtaining a therapeutic cell population; b) identifying a mammal into which therapeutic cell population is desired to be administered to; c) assessing potential for complement activation in said mammal; d) modulating said complement activation; and e) administering said therapeutic cell population.

Preferred embodiments include methods wherein said cellular therapy comprises administration of cell derived from a group comprising of: a) stem cells; b) progenitor cells; c) mesenchymal stem cells; and d) hematopoietic stem cells.

Preferred embodiments include methods wherein said stem cells are selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Preferred embodiments include methods wherein said embryonic stem cells are totipotent.

Preferred embodiments include methods wherein said embryonic stem cells express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred embodiments include methods wherein said cord blood stem cells are multipotent and capable of differentiating into endothelial, muscle, and neuronal cells.

Preferred embodiments include methods wherein said cord blood stem cells are identified based on expression of one or more antigens selected from a group comprising: SSEA-3, SSEA-4, CD9, CD34, c-kit, OCT-4, Nanog, and CXCR-4

Preferred embodiments include methods wherein said cord blood stem cells do not express one or more markers selected from a group comprising of: CD3, CD45, and CD11b.

Preferred embodiments include methods wherein said placental stem cells are isolated from the placental structure.

Preferred embodiments include methods wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

Preferred embodiments include methods wherein said bone marrow stem cells comprise of bone marrow mononuclear cells.

Preferred embodiments include methods wherein said bone marrow stem cells are selected based on the ability to differentiate into one or more of the following cell types: endothelial cells, muscle cells, and neuronal cells.

Preferred embodiments include methods wherein said bone marrow stem cells are selected based on expression of one or more of the following antigens: CD34, c-kit, flk-1, Stro-1, CD105, CD73, CD31, CD146, vascular endothelial-cadherin, CD133 and CXCR-4.

Preferred embodiments include methods wherein said bone marrow stem cells are enriched for expression of CD133.

Preferred embodiments include methods wherein said amniotic fluid stem cells are isolated by introduction of a fluid extraction means into the amniotic cavity under ultrasound guidance.

Preferred embodiments include methods wherein said amniotic fluid stem cells are selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1.

Preferred embodiments include methods wherein said amniotic fluid stem cells are selected based on lack of expression of one or more of the following antigens: CD34, CD45, and HLA Class II.

Preferred embodiments include methods wherein said neuronal stem cells are selected based on expression of one or more of the following antigens: RC-2, 3CB2, BLB, Sox-2hh, GLAST, Pax 6, nestin, Muashi-1, NCAM , A2B5 and prominin.

Preferred embodiments include methods wherein said circulating peripheral blood stem cells are characterized by ability to proliferate in vitro for a period of over 3 months.

Preferred embodiments include methods wherein said circulating peripheral blood stem cells are characterized by expression of CD34, CXCR4, CD117, CD113, and c-met.

Preferred embodiments include methods wherein said circulating peripheral blood stem cells lack substantial expression of differentiation associated markers.

Preferred embodiments include methods wherein said differentiation associated markers are selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

Preferred embodiments include methods wherein said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred embodiments include methods wherein said mesenchymal stem cells do not express substantial levels of HLA-DR, CD117, and CD45.

Preferred embodiments include methods wherein said mesenchymal stem cells are derived from a group selected of: bone marrow, adipose tissue, umbilical cord blood, placental tissue, peripheral blood mononuclear cells, differentiated embryonic stem cells, and differentiated progenitor cells.

Preferred embodiments include methods wherein said germinal stem cells express markers selected from a group comprising of: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1.

Preferred embodiments include methods wherein said adipose tissue derived stem cells express markers selected from a group comprising of: CD13, CD29, CD44, CD63, CD73, CD90, CD166, Aldehyde dehydrogenase (ALDH), and ABCG2.

Preferred embodiments include methods wherein said adipose tissue derived stem cells are a population of purified mononuclear cells extracted from adipose tissue capable of proliferating in culture for more than 1 month.

Preferred embodiments include methods wherein said exfoliated teeth derived stem cells express markers selected from a group comprising of: STRO-1, CD146 (MUC18), alkaline phosphatase, MEPE, and bFGF.

Preferred embodiments include methods wherein said hair follicle stem cells express markers selected from a group comprising of: cytokeratin 15, Nanog, and Oct-4.

Preferred embodiments include methods wherein said hair follicle stem cells are capable of proliferating in culture for a period of at least one month.

Preferred embodiments include methods wherein said hair follicle stem cells secrete one or more of the following proteins when grown in culture: basic fibroblast growth factor (bFGF), endothelin-1 (ET-1) and stem cell factor (SCF).

Preferred embodiments include methods wherein said dermal stem cells express markers selected from a group comprising of: CD44, CD13, CD29, CD90, and CD105.

Preferred embodiments include methods wherein said dermal stem cells are capable of proliferating in culture for a period of at least one month.

Preferred embodiments include methods wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred embodiments include methods wherein said reprogrammed stem cells are selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

Preferred embodiments include methods wherein said nuclear transfer comprises introducing nuclear material to a cell substantially enucleated, said nuclear material deriving from a host whose genetic profile is sought to be dedifferentiated.

Preferred embodiments include methods wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Preferred embodiments include methods wherein said DNA demethylating agent is selected from a group comprising of: 5-azacytidine, psammaplin A, and zebularine.

Preferred embodiments include methods wherein said histone deacetylase inhibitor is selected from a group comprising of: valproic acid, trichostatin-A, trapoxin A and depsipeptide.

Preferred embodiments include methods wherein said cells are identified based on expression multidrug resistance transport protein (ABCG2) or ability to efflux intracellular dyes such as rhodamine-123 and or Hoechst 33342.

Preferred embodiments include methods wherein said cells are derived from tissues such as pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

Preferred embodiments include methods wherein said committed progenitor cells are selected from a group comprising of: endothelial progenitor cells, neuronal progenitor cells, and hematopoietic progenitor cells.

Preferred embodiments include methods wherein said committed endothelial progenitor cells are purified from the bone marrow.

Preferred embodiments include methods wherein said committed endothelial progenitor cells are purified from peripheral blood.

Preferred embodiments include methods wherein said committed endothelial progenitor cells are purified from peripheral blood of a patient whose committed endothelial progenitor cells are mobilized by administration of a mobilizing agent or therapy.

Preferred embodiments include methods wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1.

Preferred embodiments include methods wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Preferred embodiments include methods wherein said committed endothelial progenitor cells express markers selected from a group comprising of: CD31, CD34, AC133, CD146 and flk1.

Preferred embodiments include methods wherein said committed hematopoietic cells are purified from the bone marrow.

Preferred embodiments include methods wherein said committed hematopoietic progenitor cells are purified from peripheral blood.

Preferred embodiments include methods wherein said committed hematopoietic progenitor cells are purified from peripheral blood of a patient whose committed hematopoietic progenitor cells are mobilized by administration of a mobilizing agent or therapy.

Preferred embodiments include methods wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1.

Preferred embodiments include methods wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Preferred embodiments include methods wherein said committed hematopoietic progenitor cells express the marker CD133.

Preferred embodiments include methods wherein said committed hematopoietic progenitor cells express the marker CD34.

Preferred embodiments include methods wherein an antioxidant is administered at a therapeutically sufficient concentration to a patient in need thereof.

Preferred embodiments include methods wherein said antioxidant is selected from a group comprising of: ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, and superoxide dismutase.

Preferred embodiments include methods wherein said antioxidant is administered prior to administration of stem cells at a concentration sufficient to reduce oxidative stress from inhibiting the beneficial effects of said stem cells function.

Preferred embodiments include methods wherein said antioxidant is administered concurrently with stem cells in order to allow maximum stem cell function.

Preferred embodiments include methods wherein said antioxidant is administered subsequent to stem cell administration in order to allow said administered stem cells to exert maximal stem cell function.

Preferred embodiments include methods wherein said drug which inhibits complement activity inhibits the formation of terminal complement or C5a.

Preferred embodiments include methods wherein said drug which inhibits formation of terminal complement or C5a is a whole antibody or an antibody fragment.

Preferred embodiments include methods wherein said whole antibody or antibody fragment is a human, humanized, chimerized or deimmunized antibody or antibody fragment.

Preferred embodiments include methods wherein said whole antibody or antibody fragment inhibits cleavage of complement C5.

Preferred embodiments include methods wherein said antibody fragment is selected from the group consisting of an Fab, an F(ab').sub.2, an Fv, a domain antibody, and a single-chain antibody.

Preferred embodiments include methods wherein said antibody fragment is pexelizumab.

Preferred embodiments include methods wherein said whole antibody is eculizumab.

Preferred embodiments include methods wherein said eculizumab is administered once every 2 weeks.

Preferred embodiments include methods wherein said inhibitor of complement activity is selected from the group consisting of a i) soluble complement receptor, ii) CD59, iii) CD55, iv) CD46, and v) an antibody to C5, C6, C7, C8, or C9.

### DETAILED DESCRIPTION OF THE INVENTION

Disclosed are means of inducing a tolerogenic state in the lung of an individual susceptible to, or, suffering from acute respiratory distress syndrome (ARDS). The invention teaches that administration of immature dendritic cells, of autologous and/or allogeneic origin, provides an environment conducive to stimulation of cells which inhibit inflammation and stimulate regeneration of damaged pulmonary cells. In one embodiment of the invention, patients are identified as having risk of ARDS based on typical clinical parameters and/or cytokine alterations.

The invention provides mean of enhancing cellular therapy efficacy by inhibition of complement activation. In one particular embodiment the invention teaches the reduction of complement activation through inhibition the classical, alternative or other pathways as a means of augmenting efficacy of mesenchymal stem cell therapy. One particular embodiment of the invention teaches enhancement of mesenchymal stem cell viability in vivo be complement depletion using agents such as cobra venom factor and/or antibodies to complement components such as C3 and/or C5. Other embodiments of the invention include maintaining therapeutic activity of mesenchymal stem cells in vivo by administration of complement inhibitors. In certain embodiments, the inhibition of complement activity is effected by chronic administration of a drug directed against complement C5. A preferred drug that inhibits complement activity is an antibody specific to one or more components of complement, for example, C5. In certain preferred embodiments, the antibody inhibits the cleavage of C5 and thereby inhibits the formation of both C5a and C5b-9. The antibody may be, e.g., a monoclonal antibody, a chimeric antibody (e.g., a humanized antibody), an antibody fragment (e.g., Fab), a single chain antibody, an Fv, or a domain antibody. Other inhibitors of complement include various agents that known to those of skill in the art. Antibodies can be made to individual components of activated complement, e.g., antibodies to C5a, C7, C9, etc. (see, e.g., U.S. Pat. No. 6,534,058; published U.S. patent application US 2003/0129187; and U.S. Pat. No. 5,660,825). Proteins are known which inhibit complement-mediated lysis, including CD59, CD55, CD46 and other inhibitors of C8 and C9 (see, e.g., U.S. Pat. No. 6,100,443). U.S. Pat. No. 6,355,245 teaches an antibody which binds to C5 and prevents it from being cleaved into C5a and C5b thereby preventing the formation not only of C5a but also the C5b-9 complex. Proteins known as complement receptors and which bind complement are also known (see, Published PCT Patent Application WO 92/10205 and U.S. Pat. No. 6,057,131). Use of soluble forms of complement receptors, e.g., soluble CR1, can inhibit the consequences of complement activation such as neutrophil oxidative burst, complement mediated hemolysis, and C3a and C5a production. Those of skill in the art recognize the above as some, but not all, of the known methods of inhibiting complement and its activation.

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents (for example: patents, patent applications, scientific publications, manufacturer's specifications, instructions, GenBank Accession Number sequence submissions etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

An “epitope”, also known as antigenic determinant, is the part of a macromolecule that is recognized by the immune system, specifically by antibodies, B cells, or T cells. As used herein, an “epitope” is the part of a macromolecule capable of binding to a compound (e.g. an antibody or antigen-binding fragment thereof) as described herein. In this context, the term “binding” preferably relates to a specific binding. Epitopes usually consist of chemically active surface groupings of molecules such as amino acids or sugar side chains and usually have specific three-dimensional structural characteristics, as well as specific charge characteristics. Conformational and non-conformational epitopes can be distinguished in that the binding to the former but not the latter is lost in the presence of denaturing solvents.

“inhibitor of C5a”, as used herein, refers to a compound that inhibits a biological activity of C5a. The term “inhibitor of C5a” particularly refers to a compound that interferes with the binding of C5a to the C5a receptors, C5aR and C5L2; especially to a compound that interferes with the binding of C5a to C5aR. Accordingly, the term “inhibitor of C5a” encompasses compounds that specifically bind to C5a and inhibit binding of C5a to C5aR as well as compounds that specifically bind to C5aR and inhibit binding of C5a to C5aR. Exemplary inhibitors of C5a include the C5a inhibitory peptide (C5aIP), the selective C5a receptor antagonists PMX53 and CCX168, and the anti-C5a antibodies disclosed in WO 2011/063980 A1 (also published as US 2012/0231008 A1). The term “inhibitor of C5a” and “C5a inhibitor” are used interchangeably herein.

“antibody” typically refers to a glycoprotein comprising at least two heavy (H) chains and two light (L) chains inter-connected by disulfide bonds, or an antigen-binding portion thereof. The term “antibody” also includes all recombinant forms of antibodies, in particular of the antibodies described herein, e.g. antibodies expressed in prokaryotes, unglycosylated antibodies, antibodies expressed in eukaryotes (e.g. CHO cells), glycosylated antibodies, and any antigen-binding antibody fragments and derivatives as described below. Each heavy chain is comprised of a heavy chain variable region (abbreviated herein as VH or V.sub.H) and a heavy chain constant region (abbreviated herein as CH or C.sub.H). The heavy chain constant region can be further subdivided into three parts, referred to as CHL CH2, and CH3 (or C.sub.H1, C.sub.H2, and C.sub.H3). Each light chain is comprised of a light chain variable region (abbreviated herein as VL or V.sub.L) and a light chain constant region (abbreviated herein as CL or C.sub.L). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

“patient” means any mammal or bird who may benefit from a treatment with an inhibitor of C5a described herein. Preferably, a “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), or primates including monkeys (e.g. African green monkeys, chimpanzees, bonobos, gorillas) and human beings. It is particularly preferred that the “patient” is a human being. The terms “patient” and “subject to be treated” (or just: “subject”) are used interchangeably herein.

“treat”, “treating” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

“carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

The complement system is described in detail in U.S. Pat. No. 6,355,245. The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum [104, 105]. Complement components achieve their immune defensive functions by interacting in a series of intricate but precise enzymatic cleavage and membrane binding events. The resulting complement cascade leads to the production of products with opsonic, immunoregulatory, and lytic functions. The complement cascade progresses via the classical pathway or the alternative pathway. These pathways share many components and, while they differ in their initial steps, they converge and share the same “terminal complement” components (C5 through C9) responsible for the activation and destruction of target cells. The classical complement pathway is typically initiated by antibody recognition of and binding to an antigenic site on a target cell. The alternative pathway is usually antibody independent and can be initiated by certain molecules on pathogen surfaces. Both pathways converge at the point where complement component C3 is cleaved by an active protease (which is different in each pathway) to yield C3a and C3b. Other pathways activating complement attack can act later in the sequence of events leading to various aspects of complement function [106]. C3a is an anaphylatoxin. C3b binds to bacterial and other cells, as well as to certain viruses and immune complexes, and tags them for removal from the circulation. C3b in this role is known as opsonin. The opsonic function of C3b is considered to be the most important anti-infective action of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone. C3b also forms a complex with other components unique to each pathway to form classical or alternative C5 convertase, which cleaves C5 into C5a and C5b. C3 is thus regarded as the central protein in the complement reaction sequence since it is essential to both the alternative and classical pathways. This property of C3b is regulated by the serum protease Factor I, which acts on C3b to produce iC3b. While still functional as opsonin, iC3b cannot form an active C5 convertase.

C5 is a 190 kDa beta globulin found in normal serum at approximately 75.mu.g/mL (0.4.mu.M.) C5 is glycosylated, with about 1.5-3 percent of its mass attributed to carbohydrate. Mature C5 is a heterodimer of a 999 amino acid 115 kDa alpha chain that is disulfide linked to a 656 amino acid 75 kDa beta chain. C5 is synthesized as a single chain precursor protein product of a single copy gene (Haviland et al., 1991). The cDNA sequence of the transcript of this gene predicts a secreted pro-05 precursor of 1659 amino acids along with an 18 amino acid leader sequence. The pro-C5 precursor is cleaved after amino acid 655 and 659, to yield the beta chain as an amino terminal fragment (amino acid residues +1 to 655) and the alpha chain as a carboxyl terminal fragment (amino acid residues 660 to 1658), with four amino acids deleted between the two.C5a is cleaved from the alpha chain of C5 by either alternative or classical C5 convertase as an amino terminal fragment comprising the first 74 amino acids of the alpha chain (i.e., amino acid residues 660-733). Approximately 20 percent of the 11 kDa mass of C5a is attributed to carbohydrate. The cleavage site for convertase action is at or immediately adjacent to amino acid residue 733. A compound that would bind at or adjacent to this cleavage site would have the potential to block access of the C5 convertase enzymes to the cleavage site and thereby act as a complement inhibitor. C5 can also be activated by means other than C5 convertase activity. Limited trypsin digestion and acid treatment can also cleave C5 and produce active C5b. C5a is another anaphylatoxin. C5b combines with C6, C7, and C8 to form the C5b-8 complex at the surface of the target cell. Upon binding of several C9 molecules, the membrane attack complex (MAC, C5b-9, terminal complement complex-TCC) is formed. When sufficient numbers of MACS insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis. As mentioned above, C3a and C5a are anaphylatoxins. These activated complement components can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract proinflammatory granulocytes to the site of complement activation.

In some embodiments of the invention, enhancement of cell therapy activity may be obtained by induction of RNA interference in order to suppress expression of the C5 gene. Previous publications have described the use of siRNA and shRNA in order to suppress C5 gene expression and are incorporated by reference [33, 107-113]. In one specific embodiment, suppression of C5 is achieved by administering a double-stranded ribonucleic acid (dsRNA) agent for inhibiting expression of complement component C5, wherein the dsRNA agent comprises a sense strand and an antisense strand. In one embodiment, the dsRNA agent comprises at least one modified nucleotide, as described below. In one aspect, the present invention provides a double stranded RNAi agent for inhibiting expression of complement component C5 wherein the double stranded RNAi agent comprises a sense strand and an antisense strand forming a double-stranded region, wherein the sense strand comprises at least 15 contiguous nucleotides differing by no more than 3 nucleotides wherein substantially all of the nucleotides of the sense strand and substantially all of the nucleotides of the antisense strand are modified nucleotides, and wherein the sense strand is conjugated to a ligand attached at the 3′-terminus.

In one embodiment, all of the nucleotides of the sense strand and all of the nucleotides of the antisense strand comprise a modification. In one embodiment, substantially all of the nucleotides of the sense strand are modified nucleotides selected from the group consisting of a 2′-O-methyl modification, a 2′-fluoro modification and a 3′-terminal deoxy-thymine (dT) nucleotide. In another embodiment, substantially all of the nucleotides of the antisense strand are modified nucleotides selected from the group consisting of a 2′-0-methyl modification, a 2′-fluoro modification and a 3′-terminal deoxy-thymine (dT) nucleotide. In another embodiment, the modified nucleotides are a short sequence of deoxy-thymine (dT) nucleotides. In another embodiment, the sense strand comprises two phosphorothioate internucleotide linkages at the 5′-terminus. In one embodiment, the antisense strand comprises two phosphorothioate internucleotide linkages at the 5′-terminus and two phosphorothioate internucleotide linkages at the 3′-terminus. In yet another embodiment, the sense strand is conjugated to one or more GalNAc derivatives attached through a branched bivalent or trivalent linker at the 3′-terminus. In one embodiment, substantially all of the nucleotides of the sense strand are modified nucleotides selected from the group consisting of a 2′-0-methyl modification, a 2′-fluoro modification and a 3′-terminal dT nucleotide. In another embodiment, substantially all of the nucleotides of the antisense strand are modified nucleotides selected from the group consisting of a 2′-O-methyl modification, a 2′-fluoro modification and a 3′-terminal dT nucleotide. In another embodiment, the modified nucleotides are a short sequence of deoxy-thymine (dT) nucleotides. In another embodiment, the sense strand comprises two phosphorothioate internucleotide linkages at the 5′-terminus. In one embodiment, the antisense strand comprises two phosphorothioate internucleotide linkages at the 5′-terminus and two phosphorothioate internucleotide linkages at the 3′-terminus. In yet another embodiment, the sense strand is conjugated to one or more GalNAc derivatives attached through a branched bivalent or trivalent linker at the 3′-terminus.

In one embodiment, the subject is human, and said human is treated with a cell therapy and an anti-complement component C5 antibody, or antigen-binding fragment thereof, to the subj ect.

In one embodiment, inhibition of C5 activity is performed together with administration of “Mesenchymal stem cells” or “MSCs” in some embodiments. These definitions refer to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLl, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In one embodiment, the MSC is administered together with antibody, or antigen-binding fragment thereof, inhibits cleavage of complement component C5 into fragments C5a and C5b. In another embodiment, the anti-complement component C5 antibody is eculizumab. In one embodiment, eculizumab is administered to the subject weekly at a dose less than about 600 mg for 4 weeks followed by a fifth dose at about one week later of less than about 900 mg, followed by a dose less than about 900 mg about every two weeks thereafter before, concurrent or after MSC therapy. In another embodiment, eculizumab is administered to the subject weekly at a dose less than about 900 mg for 4 weeks followed by a fifth dose at about one week later of less than about 1200 mg, followed by a dose less than about 1200 mg about every two weeks thereafter before, concurrent or after MSC therapy. In one embodiment, eculizumab is administered to the subject weekly at a dose less than about 900 mg for 4 weeks followed by a fifth dose at about one week later of less than about 1200 mg, followed by a dose less than about 1200 mg about every two weeks thereafter before, concurrent or after MSC therapy.

In another embodiment, eculizumab is administered to the subject weekly at a dose less than about 600 mg for 2 weeks followed by a third dose at about one week later of less than about 900 mg, followed by a dose less than about 900 mg about every two weeks thereafter before, concurrent or after MSC therapy. In another embodiment eculizumab is administered to the subject weekly at a dose less than about 600 mg for 2 weeks followed by a third dose at about one week later of less than about 600 mg, followed by a dose less than about 600 mg about every two weeks thereafter before, concurrent or after MSC therapy. In yet another embodiment, eculizumab is administered to the subject weekly at a dose less than about 600 mg for 1 week followed by a second dose at about one week later of less than about 300 mg, followed by a dose less than about 300 mg about every two weeks thereafter before, concurrent or after MSC therapy. In one embodiment, eculizumab is administered to the subject weekly at a dose less than about 300 mg for 1 week followed by a second dose at about one week later of less than about 300 mg, followed by a dose less than about 300 mg about every two weeks thereafter before, concurrent or after MSC therapy. another embodiment, the methods of the invention further include plasmapheresis or plasma exchange in the subject. In one such embodiment, eculizumab is administered to the subject at a dose less than about 600 mg or at a dose less than about 300 mg before, concurrent or after MSC therapy. In a further embodiment, the methods of the invention further include plasma infusion in the subject. In one such embodiment, eculizumab is administered to the subject at a dose less than about 300 mg before, concurrent or after MSC therapy. In one embodiment, eculizumab is administered to the subject at a dose of about 0.01 mg/kg to about 10 mg/kg or about 0.5 mg/kg to about 15 mg/kg. In another embodiment, eculizumab is administered to the subject at a dose of about 5 mg/kg to about 15 mg/kg before, concurrent or after MSC therapy. In one embodiment, eculizumab is administered to the subject at a dose selected from the group consisting of 0.5 mg/kg, 1 mg/kg, 1.5 mg/kg, 3 mg/kg, 5 mg/kg, 7 mg/kg, 10 mg/kg, and 15 mg/kg before, concurrent or after MSC therapy. In one embodiment, eculizumab is administered to the subject via an intravenous infusion. In another embodiment, eculizumab is administered to the subject subcutaneously.

Administration of the inhibitor of complement activity is performed according to methods known to those of skill in the art. These inhibitors are administered preferably before the time of allograft transplantation or at the time of transplantation with administration continuing in a chronic fashion. These inhibitors can additionally be administered during a rejection episode in the event such an episode does occur.

In the present disclosure uses of a drug that inhibits complement activity and an immunosuppressive agent in the manufacture of a medicament or medicament package are provided. Such medicament or medicament package is useful in enhancing therapeutic activity of cell therapies. In a preferred embodiment increasing activity of mesenchymal stem cell therapy. In preferred embodiments, the medicament or medicament package is formulated and prepared such that it is suitable for chronic administration, for example, stable formulations are employed. In certain embodiments, the medicament or medicament package is formulated and prepared such that it is suitable for concurrent administration of the drug that inhibits complement activity and the immunosuppressive drug to the recipient. In certain embodiments, the medicament or medicament package is formulated and prepared such that it is suitable for sequential (in either order) administration of the drug that inhibits complement activity and the immunosuppressive drug to the recipient.

A pharmaceutical package of the present disclosure may comprise a drug that inhibits complement activity and at least one cellular therap. The pharmaceutical package may further comprise a label for chronic administration. The pharmaceutical package may also comprise a label for self-administration by a patient, for example, a recipient of a cellular therapy, or instructions for a caretaker of a recipient of cellular therapy. In certain embodiments, the drug and the agent in the pharmaceutical package are in a formulation or separate formulations that are suitable for chronic administration and/or self-administration. The present disclosure also provides lyophilized formulations and formulations suitable for injection. Certain embodiments provide a lyophilized antibody formulation comprising an antibody that inhibits complement activity and a lyoprotectant. In preferred embodiments, the antibody formulation is suitable for chronic administration, for example, the antibody formulation stable. Alternative embodiments provide an injection system comprising a syringe; the syringe comprises a cartridge containing an antibody that inhibits complement activity and is in a formulation suitable for injection. An antibody employed in various embodiments of the present disclosure preferably inhibits the formation of terminal complement or C5a. In certain embodiments, antibody inhibits formation of terminal complement or C5a is a whole antibody or an antibody fragment. The whole antibody or antibody fragment may be a human, humanized, chimerized or deimmunized antibody or antibody fragment. In certain embodiments, the whole antibody or antibody fragment may inhibit cleavage of complement C5. In certain embodiments, the antibody fragment is a Fab, an F(ab′)2, an Fv, a domain antibody, or a single-chain antibody. In preferred embodiments, the antibody fragment is pexelizumab. In alternative preferred embodiments, the whole antibody is eculizumab.

In certain embodiments, a drug, such as an antibody, that inhibits complement activity is present in unit dosage form, which can be particularly suitable for self-administration. Similarly, an immunosuppressive agent of the present disclosure may also be present in unit dosage form. A formulated product of the present disclosure can be included within a container, typically, for example, a vial, cartridge, prefilled syringe or disposable pen. A doser such as the doser device described in U.S. Pat. No. 6,302,855 may also be used, for example, with an injection system of the present disclosure.

In the practice of any aspect of the present invention, a pharmaceutical composition as described herein or an inhibitor of C5a (e.g. a binding moiety specifically binding to C5a, especially hC5a, as described herein) may be administered to a patient by any route established in the art which provides a sufficient level of the inhibitor of C5a in the patient. It can be administered systemically or locally. Such administration may be parenterally, transmucosally, e.g., orally, nasally, rectally, intravaginally, sublingually, submucosally, transdermally, or by inhalation. Preferably, administration is parenteral, e.g., via intravenous or intraperitoneal injection, and also including, but is not limited to, intra-arterial, intramuscular, intradermal and subcutaneous administration. If the pharmaceutical composition of the present invention is administered locally it can be injected directly into the organ or tissue to be treated.

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered via the nasal cavity to the lungs.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

In another embodiment, for example, a drug, such as the C5a inhibitor described herein, can be delivered in a controlled-release system. For example, the inhibitor may be administered using intravenous infusion, an implantable osmotic pump, a transdermal patch, liposomes, or other modes of administration. In one embodiment, a pump may be used. In another embodiment, the compound can be delivered in a vesicle, in particular a liposome (WO 91/04014; U.S. Pat. No. 4,704,355). In another embodiment, polymeric materials can be used.

In a specific embodiment, it may be desirable to administer the pharmaceutical compositions or the C5a inhibitors of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

Selection of the preferred effective dose will be determined by a skilled artisan based upon considering several factors which will be known to one of ordinary skill in the art. Such factors include the particular form of the pharmaceutical composition, e.g. polypeptide or vector, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be prevented and/or treated or the benefit to be achieved in a normal individual, the body mass of the patient, the patient's age, the route of administration, whether administration is acute or chronic, concomitant medications, and other factors well known to affect the efficacy of administered pharmaceutical agents. Thus, the precise dosage should be decided according to the judgment of the practitioner and each patient's circumstances, e.g. depending upon the condition and the immune status of the individual patient, and according to standard clinical techniques.

For the practice of the invention, MSCs can be used together with complement inhibition for the purpose of immune modulation. The invention discloses that MSC may be viewed as a “intelligent” immune modulators. In contrast to current therapies, which globally cause immune suppression, production of anti-inflammatory factors by MSC appears to be dependent on their environment, with upregulation of factors such as TGF-b, HLA-G, IL-10, and neuropilin-A ligands galectin-1 and Semaphorin-3A in response to immune/inflammatory stimuli but little in the basal state [114-118]. This property may be selected for when utilizing the marker combinations disclosed in the current invention. Additionally, the invention discloses synergies between complement inhibition and MSC administration of induction of immune modulation and/or tolerogenesis. The combined use of MSC and complement inhibition may be directed towards conditions such as autoimmunity, transplant rejection, inflammation, sepsis, ARDS and acute radiation syndrome.

Additionally, systemically administered MSC possess ability to selectively home to injured/hypoxic areas by recognition of signals such as HMGB1 or CXCR1, respectively [119-122]. The ability to home to injury, combined with selective induction of immune modulation only in response to inflammatory/danger signals suggests the possibility that systemically administered MSC do not cause global immune suppression. This is supported by clinical studies using MSC for other inflammatory conditions, which to date, have not reported immune suppression associated adverse effects [123-125]. Another important aspect of MSC therapy is their ability to regenerate injured tissue through direct differentiation into articular tissue [126], as well as ability to secret growth factors capable of augmenting endogenous regenerative processes [127].

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

In one embodiment MSC donor lots are generated from umbilical cord tissue. Means of generating umbilical cord tissue MSC have been previously published and are incorporated by reference [128-134]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

In one embodiment, bone marrow MSC lots are generated, means of generating BM MSC are known in the literature and examples are incorporated by reference.

In one embodiment BM-MSC are generated as follows:

1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.

2. The total number of nucleated cells in the BM sample is counted by taking 10 .mu.L BM and diluting it 1/50-1/100 with 3% Acetic Acid with Methylene Blue (STEMCELL Catalog #07060). Cells are counted using a hemacytometer.

3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).

4. In three 50 mL conical tubes (BD Catalog #352070), 17 mL Ficoll-Paque™. PLUS (Catalog #07907/07957) is pipetted into each tube. About 23 mL of the diluted BM from step 3 was carefully layered on top of the Ficoll-Paque™. PLUS in each tube.

5. The tubes are centrifuged at room temperature (15-25.degree. C.) for 30 minutes at 300.times.g in a bench top centrifuge with the brake off.

6. The upper plasma layer is removed and discarded without disturbing the plasma:Ficoll-Paque™. PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.

7. Cells were centrifuged at 300.times.g for 10 minutes at room temperature in a bench top centrifuge with the brake on. The supernatant is removed and the cell pellet resuspended in 1-2 mL cold Isolation Buffer.

8. Cells were diluted 1/50 in 3% Acetic Acid with Methylene Blue and the total number of nucleated cells counted using a hemacytometer.

9. Cells are diluted in Complete Human MesenCult®-Proliferation medium (STEMCELL catalog #05411) at a final concentration of 1.times.10.sup.6 cells/mL.

10. BM-derived cells were ready for expansion and CFU-F assays in the presence of GW2580, which can then be used for specific applications.

### REFERENCES

1. Walport, M. J., Complement. First of two parts. N Engl J Med, 2001. 344(14): p. 1058-66.
2. Snyderman, R., H. Gewurz, and S. E. Mergenhagen, Interactions of the complement system with endotoxic lipopolysaccharide. Generation of a factor chemotactic for polymorphonuclear leukocytes. J Exp Med, 1968. 128(2): p. 259-75.
3. Haddad, A. and A. M. Wilson, Biochemistry, Complement, in StatPearls. 2020: Treasure Island (FL).
4. Lehnardt, S., Innate immunity and neuroinflammation in the CNS: the role of microglia in Toll-like receptor-mediated neuronal injury. Glia, 2010. 58(3): p. 253-63.
5. Dunkelberger, J. R. and W. C. Song, Complement and its role in innate and adaptive immune responses. Cell Res, 2010. 20(1): p. 34-50.
6. Morgan, E. L., et al., Anaphylatoxin-mediated regulation of the immune response. II. C5a-mediated enhancement of human humoral and T cell-mediated immune responses. J Immunol, 1983. 130(3): p. 1257-61.
7. Nataf, S., et al., Human T cells express the C5a receptor and are chemoattracted to C5a. J Immunol, 1999. 162(7): p. 4018-23.
8. Marsh, J. E., et al., The allogeneic T and B cell response is strongly dependent on complement components C3 and C4. Transplantation, 2001. 72(7): p. 1310-8.
9. Kim, A. H., et al., Complement C5a receptor is essential for the optimal generation of antiviral CD8+ T cell responses. J. Immunol, 2004. 173(4): p. 2524-9.
10. Fang, C., et al., Complement-dependent enhancement of CD8+ T cell immunity to lymphocytic choriomeningitis virus infection in decay-accelerating factor-deficient mice. J. Immunol, 2007. 179(5): p. 3178-86.
11. Wang, H., et al., Inhibition of terminal complement components in presensitized transplant recipients prevents antibody-mediated rejection leading to long-term graft survival and accommodation. J. Immunol, 2007. 179(7): p. 4451-63.
12. Lalli, P. N., et al., Decay accelerating factor can control T cell differentiation into IFN-gamma-producing effector cells via regulating local C5a-induced IL-12 production. J. Immunol, 2007. 179(9): p. 5793-802.
13. Strainic, M. G., et al., Locally produced complement fragments C5a and C3a provide both costimulatory and survival signals to naive CD4+ T cells. Immunity, 2008. 28(3): p. 425-35.
14. Liu, J., et al., IFN-gamma and IL-17 production in experimental autoimmune encephalomyelitis depends on local APC-T cell complement production. J. Immunol, 2008. 180(9): p. 5882-9.
15. Hegde, G. V., et al., A conformationally-biased, response-selective agonist of C5a acts as a molecular adjuvant by modulating antigen processing and presentation activities of human dendritic cells. Int Immunopharmacol, 2008. 8(6): p. 819-27.
16. Lalli, P. N., et al., Locally produced C5a binds to T cell-expressed C5aR to enhance effector T-cell expansion by limiting antigen-induced apoptosis. Blood, 2008. 112(5): p. 1759-66.
17. Pavlov, V., et al., Donor deficiency of decay-accelerating factor accelerates murine T cell-mediated cardiac allograft rejection. J Immunol, 2008. 181(7): p. 4580-9.
18. Fang, C., et al., Complement promotes the development of inflammatory T-helper 17 cells through synergistic interaction with Toll-like receptor signaling and interleukin-6 production. Blood, 2009. 114(5): p. 1005-15.
19. Raedler, H., et al., Primed CD8(+) T-cell responses to allogeneic endothelial cells are controlled by local complement activation. Am J Transplant, 2009. 9(8): p. 1784-95.
20. Li, Q., et al., The complement inhibitor FUT-175 suppresses T cell autoreactivity in experimental autoimmune encephalomyelitis. Am J Pathol, 2009. 175(2): p. 661-7.
21. Li, Q., et al., Augmenting DAF levels in vivo ameliorates experimental autoimmune encephalomyelitis. Mol Immunol, 2009. 46(15): p. 2885-91.
22. Xu, R., et al., Complement C5a regulates IL-17 by affecting the crosstalk between DC and gammadelta T cells in CLP-induced sepsis. Eur J Immunol, 2010. 40(4): p. 1079-88.
23. Hashimoto, M., et al., Complement drives Th17 cell differentiation and triggers autoimmune arthritis. J Exp Med, 2010. 207(6): p. 1135-43.
24. Chen, G., et al., Blockade of complement activation product C5a activity using specific antibody attenuates intestinal damage in trinitrobenzene sulfonic acid induced model of colitis. Lab Invest, 2011. 91(3): p. 472-83.
25. Han, G., et al., gammadeltaT-cell function in sepsis is modulated by C5a receptor signalling. Immunology, 2011. 133(3): p. 340-9.
26. Raedler, H., et al., Anti-complement component C5 mAb synergizes with CTLA4Ig to inhibit alloreactive T cells and prolong cardiac allograft survival in mice. Am J Transplant, 2011. 11(7): p. 1397-406.
27. Vieyra, M., et al., Complement regulates CD4 T-cell help to CD8 T cells required for murine allograft rejection. Am J Pathol, 2011. 179(2): p. 766-74.
28. Fusakio, M. E., et al., C5a regulates NKT and NK cell functions in sepsis. J Immunol, 2011. 187(11): p. 5805-12.
29. Mashruwala, M. A., et al., A defect in the synthesis of Interferon-gamma by the T cells of Complement-C5 deficient mice leads to enhanced susceptibility for tuberculosis. Tuberculosis (Edinb), 2011. 91 Suppl 1: p. S82-9.
30. Strainic, M. G., et al., Absence of signaling into CD4(+) cells via C3aR and CSaR enables autoinductive TGF-beta1 signaling and induction of Foxp3(+) regulatory T cells. Nat Immunol, 2013. 14(2): p. 162-71.
31. Liu, T., et al., An essential role for CSaR signaling in the optimal induction of a malaria-specific CD4+ T cell response by a whole-killed blood-stage vaccine. J Immunol, 2013. 191(1): p. 178-86.
32. Ma, N., et al., C5a regulates IL-12+ DC migration to induce pathogenic Th1 and Th17 cells in sepsis. PLoS One, 2013. 8(7): p. e69779.
33. Cravedi, P., et al., Immune cell-derived C3a and C5a costimulate human T cell alloimmunity. Am J

Transplant, 2013. 13(10): p. 2530-9.
34. Yamanaka, K., et al., Depression of Complement Regulatory Factors in Rat and Human Renal Grafts Is Associated with the Progress of Acute T-Cell Mediated Rejection. PLoS One, 2016. 11(2): p. e0148881.
35. Steinman, R. M., Dendritic cells: understanding immunogenicity. Eur J Immunol, 2007. 37 Suppl 1: p. S53-60.
36. Levings, M. K., et al., Differentiation of Tr1 cells by immature dendritic cells requires IL-10 but not CD25+ CD4+ Tr cells. Blood, 2005. 105(3): p. 1162-9.
37. Ghiringhelli, F., et al., Tumor cells convert immature myeloid dendritic cells into TGF-beta-secreting cells inducing CD4+ CD25+ regulatory T cell proliferation. J Exp Med, 2005. 202(7): p. 919-29.
38. Stepkowski, S. M., et al., Immature syngeneic dendritic cells potentiate tolerance to pancreatic islet allografts depleted of donor dendritic cells in microgravity culture condition. Transplantation, 2006. 82(12): p. 1756-63.
39. Jin, Y., et al., Induction of auto-reactive regulatory T cells by stimulation with immature autologous dendritic cells. Immunol Invest, 2007. 36(2): p. 213-32.
40. Gandhi, R., D. E. Anderson, and H. L. Weiner, Cutting Edge: Immature human dendritic cells express latency-associated peptide and inhibit T cell activation in a TGF-beta-dependent manner. J Immunol, 2007. 178(7): p. 4017-21.
41. Ureta, G., et al., Generation of dendritic cells with regulatory properties. Transplant Proc, 2007. 39(3): p. 633-7.
42. Yamazaki, S., et al., Dendritic cells are specialized accessory cells along with TGF-for the differentiation of Foxp3+ CD4+ regulatory T cells from peripheral Foxp3 precursors. Blood, 2007. 110(13): p. 4293-302.
43. Gaudreau, S., et al., Granulocyte-macrophage colony-stimulating factor prevents diabetes development in NOD mice by inducing tolerogenic dendritic cells that sustain the suppressive function of CD4+ CD25+ regulatory T cells. J Immunol, 2007. 179(6): p. 3638-47.
44. Wu, K., et al., Suppression of allergic inflammation by allergen-DNA-modified dendritic cells depends on the induction of Foxp3+ Regulatory T cells. Scand J Immunol, 2008. 67(2): p. 140-51.
45. Sarris, M., et al., Neuropilin-1 expression on regulatory T cells enhances their interactions with dendritic cells during antigen recognition. Immunity, 2008. 28(3): p. 402-13.
46. Zhang, X., et al., Generation of therapeutic dendritic cells and regulatory T cells for preventing allogeneic cardiac graft rejection. Clin Immunol, 2008. 127(3): p. 313-21.
47. Cools, N., et al., Immunosuppression induced by immature dendritic cells is mediated by TGF-beta/IL-10 double-positive CD4+ regulatory T cells. J Cell Mol Med, 2008. 12(2): p. 690-700.
48. Wang, L., et al., Programmed death 1 ligand signaling regulates the generation of adaptive Foxp3+ CD4+ regulatory T cells. Proc Natl Acad Sci U S A, 2008. 105(27): p. 9331-6.
49. Marguti, I., et al., Expansion of CD4+ CD25+ Foxp3+ T cells by bone marrow-derived dendritic cells. Immunology, 2009. 127(1): p. 50-61.
50. Qadura, M., et al., Reduction of the immune response to factor VIII mediated through tolerogenic factor VIII presentation by immature dendritic cells. J Thromb Haemost, 2008. 6(12): p. 2095-104.
51. Kuo, Y. R., et al., Alloantigen-pulsed host dendritic cells induce T-cell regulation and prolong allograft survival in a rat model of hindlimb allotransplantation. J Surg Res, 2009. 153(2): p. 317-25.
52. Bamboat, Z. M., et al., Human liver dendritic cells promote T cell hyporesponsiveness. J Immunol, 2009. 182(4): p. 1901-11.
53. Jin, C. J., et al., All-trans retinoic acid inhibits the differentiation, maturation, and function of human monocyte-derived dendritic cells. Leuk Res, 2010. 34(4): p. 513-20.
54. Zahorchak, A. F., G. Raimondi, and A. W. Thomson, Rhesus monkey immature monocyte-derived dendritic cells generate alloantigen-specific regulatory T cells from circulating CD4+ CD127-/lo T cells. Transplantation, 2009. 88(9): p. 1057-64.
55. Kushwah, R., et al., Apoptotic dendritic cells induce tolerance in mice through suppression of dendritic cell maturation and induction of antigen-specific regulatory T cells. J Immunol, 2009. 183(11): p. 7104-18.
56. Dai, H., et al., Programmed death-1 signaling is essential for the skin allograft protection by alternatively activated dendritic cell infusion in mice. Transplantation, 2009. 88(7): p. 864-73.
57. Wang, L., Adaptive Treg generation by DCs and their functional analysis. Methods Mol Biol, 2010. 595: p. 403-12.
58. Schildknecht, A., et al., FoxP3+ regulatory T cells essentially contribute to peripheral CD8+ T-cell tolerance induced by steady-state dendritic cells. Proc Natl Acad Sci U S A, 2010. 107(1): p. 199-203.
59. Heng, Y., et al., Adoptive transfer of FTY720-treated immature BMDCs significantly prolonged cardiac allograft survival. Transpl Int, 2010. 23(12): p. 1259-70.
60. Muller, T., et al., Iloprost has potent anti-inflammatory properties on human monocyte-derived dendritic cells. Clin Exp Allergy, 2010. 40(8): p. 1214-21.
61. Kong, W., J. H. Yen, and D. Ganea, Docosahexaenoic acid prevents dendritic cell maturation, inhibits antigen-specific Th1/Th17 differentiation and suppresses experimental autoimmune encephalomyelitis. Brain Behav Immun, 2011. 25(5): p. 872-82.
62. Takeda, M., et al., Oral administration of an active form of vitamin D3 (calcitriol) decreases atherosclerosis in mice by inducing regulatory T cells and immature dendritic cells with tolerogenic functions. Arterioscler Thromb Vasc Biol, 2010. 30(12): p. 2495-503.
63. Stary, G., et al., Glucocorticosteroids modify Langerhans cells to produce TGF-beta and expand regulatory T cells. J Immunol, 2011. 186(1): p. 103-12.
64. Pletinckx, K., et al., Role of dendritic cell maturity/costimulation for generation, homeostasis, and suppressive activity of regulatory T cells. Front Immunol, 2011. 2: p. 39.
65. Wolfle, S. J., et al., PD-L1 expression on tolerogenic APCs is controlled by STAT-3. Eur J Immunol, 2011. 41(2): p. 413-24.
66. Schuler, P. J., et al., Dendritic cell generation and CD4+ CD25high FOXP3+ regulatory t cells in human head and neck carcinoma during radio-chemotherapy. Eur J Med Res, 2011. 16(2): p. 57-62.
67. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
68. Amaral, M. M., et al., Thioperamide induces CD4 CD25 Foxp3 regulatory T lymphocytes in the lung mucosa of allergic mice through its action on dendritic cells. J Asthma Allergy, 2011. 4: p. 93-102.
69. Presicce, P., et al., Myeloid dendritic cells isolated from tissues of SIV-infected Rhesus macaques promote the induction of regulatory T cells. AIDS, 2012. 26(3): p. 263-73.
70. Wang, G. Y., et al., Rapamycin combined with donor immature dendritic cells promotes liver allograft survival in association with CD4(+) CD25(+) Foxp3(+) regulatory T cell expansion. Hepatol Res, 2012. 42(2): p. 192-202.
71. Petzold, C., et al., Targeted antigen delivery to DEC-205(+) dendritic cells for tolerogenic vaccination. Rev Diabet Stud, 2012. 9(4): p. 305-18.
72. Wang, G. Y., et al., Rapamycin combined with allogenic immature dendritic cells selectively expands CD4+ CD25+ Foxp3+ regulatory T cells in rats. Hepatobiliary Pancreat Dis Int, 2012. 11(2): p. 203-8.
73. Zhou, F., et al., Immune tolerance induced by intravenous transfer of immature dendritic cells via up-regulating numbers of suppressive IL-10(+) IFN-gamma(+)-producing CD4(+) T cells. Immunol Res, 2013. 56(1): p. 1-8.
74. Volchenkov, R., et al., Type 1 regulatory T cells and regulatory B cells induced by tolerogenic dendritic cells. Scand J Immunol, 2013. 77(4): p. 246-54.
75. Zhang, G., et al., Triptolide-conditioned dendritic cells induce allospecific T-cell regulation and prolong renal graft survival. J Invest Surg, 2013. 26(4): p. 191-9.
76. Farias, A. S., et al., Vitamin D3 induces IDO+ tolerogenic DCs and enhances Treg, reducing the severity of EAE. CNS Neurosci Ther, 2013. 19(4): p. 269-77.
77. Gao, X. W., et al., Mechanism of immune tolerance induced by donor derived immature dendritic cells in rat high-risk corneal transplantation. Int J Ophthalmol, 2013. 6(3): p. 269-75.
78. Lindenberg, J. J., et al., IL-10 conditioning of human skin affects the distribution of migratory dendritic cell subsets and functional T cell differentiation. PLoS One, 2013. 8(7): p. e70237.
79. Huang, Y., et al., Increased expression of herpesvirus entry mediator in 1,25-dihydroxyvitamin D3-treated mouse bone marrow-derived dendritic cells promotes the generation of CD4(+)CD25(+)Foxp3(+) regulatory T cells. Mol Med Rep, 2014. 9(3): p. 813-8.
80. Pletinckx, K., et al., Immature dendritic cells convert anergic nonregulatory T cells into Foxp3- IL-10+ regulatory T cells by engaging CD28 and CTLA-4. Eur J Immunol, 2015. 45(2): p. 480-91.
81. Wei, Y., et al., Infusion of dendritic cells carrying donor lymphocytes treated with 8-methoxypsoralen and ultraviolet A light induces CD19+ IL-10+ regulatory B cells and promotes skin allograft survival. Transplant Proc, 2014. 46(10): p. 3641-6.
82. Dong, M., et al., Rapamycin Combined with Immature Dendritic Cells Attenuates Obliterative Bronchiolitis in Trachea Allograft Rats by Regulating the Balance of Regulatory and Effector T Cells. Int Arch Allergy Immunol, 2015. 167(3): p. 177-85.
83. Bergmann, C., et al., Expansion and characteristics of human T regulatory type 1 cells in co-cultures simulating tumor microenvironment. Cancer Immunol Immunother, 2007. 56(9): p. 1429-42.
84. Kaporis, H. G., et al., Human basal cell carcinoma is associated with Foxp3+ T cells in a Th2 dominant microenvironment. J Invest Dermatol, 2007. 127(10): p. 2391-8.
85. Koido, S., et al., In vitro generation of cytotoxic and regulatory T cells by fusions of human dendritic cells and hepatocellular carcinoma cells. J Transl Med, 2008. 6: p. 51.
86. Li, L., et al., Hepatoma cells inhibit the differentiation and maturation of dendritic cells and increase the production of regulatory T cells. Immunol Lett, 2007. 114(1): p. 38-45.
87. Shi, D., et al., Artificial phosphatidylserine liposome mimics apoptotic cells in inhibiting maturation and immunostimulatory function of murine myeloid dendritic cells in response to 1-chloro-2,4-dinitrobenze in vitro. Arch Dermatol Res, 2007. 299(7): p. 327-36.
88. Kranich, J., et al., Follicular dendritic cells control engulfment of apoptotic bodies by secreting Mfge8. J Exp Med, 2008. 205(6): p. 1293-302.
89. Rodriguez-Manzanet, R., et al., T and B cell hyperactivity and autoimmunity associated with niche-specific defects in apoptotic body clearance in TIM-4-deficient mice. Proc Natl Acad Sci U S A, 2010. 107(19): p. 8706-11.
90. Frey, B. and U. S. Gaipl, The immune functions of phosphatidylserine in membranes of dying cells and microvesicles. Semin Immunopathol, 2011. 33(5): p. 497-516.
91. Saas, P., et al., Phosphatidylserine-expressing cell by-products in transfusion: A pro-inflammatory or an anti-inflammatory effect? Transfus Clin Biol, 2012. 19(3): p. 90-7.
92. Trahtemberg, U. and D. Mevorach, Apoptotic Cells Induced Signaling for Immune Homeostasis in Macrophages and Dendritic Cells. Front Immunol, 2017. 8: p. 1356.
93. Perruche, S., et al., CD3-specific antibody-induced immune tolerance involves transforming growth factor-beta from phagocytes digesting apoptotic T cells. Nat Med, 2008. 14(5): p. 528-35.
94. Dumitriu, I. E., et al., Human dendritic cells produce TGF-beta 1 under the influence of lung carcinoma cells and prime the differentiation of CD4+ CD25+ Foxp3+ regulatory T cells. J Immunol, 2009. 182(5): p. 2795-807.
95. Ragni, M. V., et al., Factor VIII-pulsed dendritic cells reduce anti-factor VIII antibody formation in the hemophilia A mouse model. Exp Hematol, 2009. 37(6): p. 744-54.
96. Kushwah, R., et al., Uptake of apoptotic DC converts immature DC into tolerogenic DC that induce differentiation of Foxp3+ Treg. Eur J Immunol, 2010. 40(4): p. 1022-35.
97. Zheng, D. H., et al., Uptake of donor lymphocytes treated with 8-methoxypsoralen and ultraviolet A light by recipient dendritic cells induces CD4+ CD25+ Foxp3+ regulatory T cells and down-regulates cardiac allograft rejection. Biochem Biophys Res Commun, 2010. 395(4): p. 540-6.
98. Carrascal, M. A., et al., Sialyl Tn-expressing bladder cancer cells induce a tolerogenic phenotype in innate and adaptive immune cells. Mol Oncol, 2014. 8(3): p. 753-65.
99. Lutz, M. B., Induction of CD4(+) Regulatory and Polarized Effector/helper T Cells by Dendritic Cells. Immune Netw, 2016. 16(1): p. 13-25.
100. Verbovetski, I., et al., Opsonization of apoptotic cells by autologous iC3b facilitates clearance by immature dendritic cells, down-regulates DR and CD86, and up-regulates CC chemokine receptor 7. J Exp Med, 2002. 196(12): p. 1553-61.
101. Fadok, V. A., et al., Macrophages that have ingested apoptotic cells in vitro inhibit proinflammatory cytokine production through autocrine/paracrine mechanisms involving TGF-beta, PGE2, and PAF. J Clin Invest, 1998. 101(4): p. 890-8.
102. McDonald, P. P., et al., Transcriptional and translational regulation of inflammatory mediator production by endogenous TGF-beta in macrophages that have ingested apoptotic cells. J Immunol, 1999. 163(11): p. 6164-72.
103. Stuart, L. M., et al., Inhibitory effects of apoptotic cell ingestion upon endotoxin-driven myeloid dendritic cell maturation. J Immunol, 2002. 168(4): p. 1627-35.
104. Woo, J. J., et al., The complement system in schizophrenia: where are we now and what's next? Mol Psychiatry, 2020. 25(1): p. 114-130.
105. McGeer, P. L., M. Lee, and E. G. McGeer, A review of human diseases caused or exacerbated by aberrant complement activation. Neurobiol Aging, 2017. 52: p. 12-22.
106. Sim, R. B. and A. Laich, Serine proteases of the complement system. Biochem Soc Trans, 2000. 28(5): p. 545-50.
107. Cheng, L. L., et al., [Effect of C5-siRNA silencing receptor C5 on myocardial ischemia injury in rats]. Nan Fang Yi Ke Da Xue Xue Bao, 2010. 30(6): p. 1486-8.
108. Ebrahimi, K. B., et al., Decreased membrane complement regulators in the retinal pigmented epithelium contributes to age-related macular degeneration. J Pathol, 2013. 229(5): p. 729-42.
109. Bora, N. S., et al., Complement activation via alternative pathway is critical in the development of laser-induced choroidal neovascularization: role of factor 8 and factor H. J Immunol, 2006. 177(3): p. 1872-8.
110. Tang, K., et al., Protective effect of C5 shRNA on myocardial ischemia-reperfusion injury in rats. Can J Physiol Pharmacol, 2012. 90(10): p. 1394-402.
111. Hattori, Y., et al., Transdermal Delivery of Small Interfering RNA with Elastic Cationic Liposomes in Mice. J Pharm (Cairo), 2013. 2013: p. 149695.
112. Mehta, G., et al., A New Approach for the Treatment of Arthritis in Mice with a Novel Conjugate of an Anti-C5aR1 Antibody and C5 Small Interfering RNA. J Immunol, 2015. 194(11): p. 5446-54.
113. Kusner, L. L., et al., Investigational RNAi Therapeutic Targeting C5 Is Efficacious in Pre-clinical Models of Myasthenia Gravis. Mol Ther Methods Clin Dev, 2019. 13: p. 484-492.
114. Nasef, A., et al., Selected Stro-1-enriched bone marrow stromal cells display a major suppressive effect on lymphocyte proliferation. Int J Lab Hematol, 2009. 31(1): p. 9-19.
115. Nasef, A., et al., Leukemia inhibitory factor: Role in human mesenchymal stem cells mediated immunosuppression. Cell Immunol, 2008. 253(1-2): p. 16-22.
116. Lepelletier, Y., et al., Galectin-1 and Semaphorin-3A are two soluble factors conferring T cell immunosuppression to bone marrow mesenchymal stem cell. Stem Cells Dev, 2009.
117. Renner, P., et al., Mesenchymal stem cells require a sufficient, ongoing immune response to exert their immunosuppressive function. Transplant Proc, 2009. 41(6): p. 2607-11.
118. Ryan, J. M., et al., Interferon-gamma does not break, but promotes the immunosuppressive capacity of adult human mesenchymal stem cells. Clin Exp Immunol, 2007. 149(2): p. 353-63.
119. Lisheng, W., E. Meng, and Z. Guo, High mobility group box 1 protein inhibits the proliferation of human mesenchymal stem cells and promotes their migration and differentiation along osteoblastic pathway. Stem Cells Dev, 2008.
120. Kitaori, T., et al., Stromal cell-derived factor 1/CXCR4 signaling is critical for the recruitment of mesenchymal stem cells to the fracture site during skeletal repair in a mouse model. Arthritis Rheum, 2009. 60(3): p. 813-23.
121. Wang, Y., Y. Deng, and G. Q. Zhou, SDF-1alpha/CXCR4-mediated migration of systemically transplanted bone marrow stromal cells towards ischemic brain lesion in a rat model. Brain Res, 2008. 1195: p. 104-12.
122. Shi, M., et al., Regulation of CXCR4 expression in human mesenchymal stem cells by cytokine treatment: role in homing efficiency in NOD/SCID mice. Haematologica, 2007. 92(7): p. 897-904.
123. Gunzberg, W. H. and B. Salmons, Stem cell therapies: on track but suffer setback. Curr Opin Mol Ther, 2009. 11(4): p. 360-3.
124. Kebriaei, P., et al., Adult human mesenchymal stem cells added to corticosteroid therapy for the treatment of acute graft-versus-host disease. Biol Blood Marrow Transplant, 2009. 15(7): p. 804-11.
125. Dryden, G. W., Overview of stem cell therapy for Crohn's disease. Expert Opin Biol Ther, 2009. 9(7): p. 841-7.
126. Richardson, S. M., et al., Mesenchymal stem cells in regenerative medicine: opportunities and challenges for articular cartilage and intervertebral disc tissue engineering. J Cell Physiol. 222(1): p. 23-32.
127. Bouffi, C., et al., Multipotent mesenchymal stromal cells and rheumatoid arthritis: risk or benefit? Rheumatology (Oxford), 2009. 48(10): p. 1185-9.
128. Van Pham, P., et al., Isolation and proliferation of umbilical cord tissue derived mesenchymal stem cells for clinical applications. Cell Tissue Bank, 2015.
129. Fazzina, R., et al., A new standardized clinical-grade protocol for banking human umbilical cord tissue cells. Transfusion, 2015. 55(12): p. 2864-73.
130. Bieback, K., Platelet lysate as replacement for fetal bovine serum in mesenchymal stromal cell cultures. Transfus Med Hemother, 2013. 40(5): p. 326-35.
131. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.
132. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.
133. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.
134. Friedman, R., et al., Umbilical cord mesenchymal stem cells: adjuvants for human cell transplantation. Biol Blood Marrow Transplant, 2007. 13(12): p. 1477-86.

1. A method of enhancing efficacy of cellular therapy comprising the steps of: a) obtaining a therapeutic cell population; b) identifying a mammal into which therapeutic cell population is desired to be administered to; c) assessing potential for complement activation in said mammal; d) modulating said complement activation by administering a drug to said mammal; and e) administering said therapeutic cell population to said mammal.

2. The method of claim 1, wherein said therapeutic cell population is selected from the group consisting of: a) stem cells; b) progenitor cells; c) mesenchymal stem cells; and d) hematopoietic stem cells.

3. The method of claim 2, wherein said stem cells are selected from the group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells, and side population stem cells.

4. The method of claims 1 wherein an antioxidant is administered at a therapeutically sufficient concentration to said mammal.

5. The method of claim 4, wherein said antioxidant is selected from the group consisting of: ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, and superoxide dismutase.

6. The method of claim 1, wherein said drug inhibits the formation of terminal complement or C5a.

7. The method of claim 6, wherein said drug inhibits formation of terminal complement or C5a and is a whole antibody or an antibody fragment.

8. The method of claim 7, wherein said whole antibody or antibody fragment is selected from the group consisting of: human, humanized, chimerized or deimmunized antibody or antibody fragment.

9. The method of claim 8, wherein said whole antibody or antibody fragment inhibits cleavage of complement C5.

10. The method of claim 8, wherein said antibody fragment is selected from the group consisting of an Fab, an F(ab′).sub.2, an Fv, a domain antibody, and a single-chain antibody.

11. The method of claim 8, wherein said antibody fragment is pexelizumab.

12. The method of claim 8, wherein said whole antibody is eculizumab.

13. The method claim 12, wherein said eculizumab is administered once every 2 weeks.

14. The method of claim 1 wherein said drug is an inhibitor of complement activity and is selected from the group consisting of: i) soluble complement receptor, ii) CD59, iii) CD55, iv) CD46, and v) an antibody to C5, C6, C7, C8, or C9.

15. The method of claim 1, wherein said drug modulating complement activation augments in vivo activity of said stem cell to secrete anti-apoptotic factors.

16. The method of claim 1, wherein said drug modulating complement activation augments in vivo activity of said stem cell to secrete angiogenic factors.

17. The method of claim 1, wherein said drug modulating complement activation augments in vivo activity of said stem cell to secrete PDGF-BB.

18. The method of claim 1, wherein said drug modulating complement activation augments in vivo activity of said stem cell to increase T regulatory cell activity.

19. The method of claim 1, wherein said drug modulating complement activation augments in vivo activity of said stem cell to block dendritic cell maturation.

20. The method of claim 1, wherein said drug modulating complement activation augments in vivo activity of said stem cell to induce generation of M2 macrophages.
